(19) United States
(12) Patent Application Publication
(10) Pub. No.: US 2024/0197754 A1
(43) Pub. Date: Jun. 20, 2024

(54) ANTICONVULSANT ACTIVITY OF STEROIDS

(71) Applicant: THE REGENTS OF THE UNIVERSITY OF CALIFORNIA, Oakland, CA (US)

(72) Inventors: MICHAEL A. ROGAWSKI, Sacramento, CA (US); Dorota ZOLKOWSKA, Davis, CA (US)

(21) Appl. No.: 18/442,086

(22) Filed: Feb. 14, 2024

Related U.S. Application Data
(63) Continuation of application No. 17/064,517, filed on Oct. 6, 2020, now abandoned.
Application No. 17/064,517 is a continuation of application No. 16/241,877, filed on Jan. 7, 2019, now abandoned.
Application No. 16/241,877 is a continuation of application No. 15/632,360, filed on Jun. 25, 2017, now abandoned.
Application No. 15/632,360 is a continuation of application No. 14/646,886, filed on May 22, 2015, now abandoned.
(60) Provisional application No. 61/732,252, filed on Nov. 30, 2012.

(51) Int. Cl. A61K 31/57; A61K 9/00; A61K 9/08; A61K 31/56; A61K 31/573; A61K 47/40

(52) U.S. Cl. CPC A61K 31/57; A61K 9/0019; A61K 9/08; A61K 31/56; A61K 31/573; A61K 47/40

(57) ABSTRACT

The present invention relates to methods of preventing, inhibiting, delaying. and/or mitigating seizures by administration of a steroid, e.g., a neurosteroid, e.g., allopregnanolone.

### CROSS-REFERENCE TO RELATED APPLICATIONS

This application is a continuation of U.S. application Ser. No. 17/064,517, filed on Oct. 6, 2020, which is a continuation of U.S. application Ser. No. 16/241,877, filed on Jan. 7, 2019, which is a continuation of U.S. application Ser. No. 15/632,360, filed on Jun. 25, 2017, which is a continuation of U.S. application Ser. No. 14/646,886, filed on May 22, 2015, which is a U.S. national phase application under 35 U.S.C. § 371 of International Appl. No. PCT/US2013/072351, filed on Nov. 27, 2013, which claims the benefit under 35 U.S.C. § 119(e) of U.S. Provisional Application No. 61/732,252, filed on Nov. 30, 2012, all of which are hereby incorporated herein by reference in their entireties for all purposes.

### FIELD

The present invention relates to methods of preventing, inhibiting, delaying, and/or mitigating seizures by administration of a steroid, e.g., a neurosteroid, e.g., allopregnanolone.

### BACKGROUND

Steroids, including neurosteroids (e.g., allopregnanolone) are highly insoluble in aqueous solution. Various approaches are used to enhance aqueous dissolution, including the use of cyclodextrin solutions. However, even with cyclodextrin as a solvation aid, solubility is not sufficient to permit systemic delivery for the treatment of medical conditions.

### SUMMARY

In one aspect, methods of preventing, treating, reducing, and/or mitigating one or more symptoms associated with and/or caused by traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, and smoking cessation in a subject in need thereof are provided. In some embodiments, the methods comprise administering to the subject a steroid.

In another aspect, methods of preventing, treating, reducing, and/or mitigating symptoms associated with and/or caused by epilepsy, in a subject in need thereof are provided. In some embodiments, the methods comprise administering to the subject a steroid.

In a further aspect, methods of accelerating the termination or abortion of an impending seizure in a subject in need thereof are provided. In some embodiments, the methods comprise administering to the subject a steroid.

With respect to embodiments of the methods, in some embodiments, the steroid is a neurosteroid. In some embodiments, the neurosteroid is selected from the group consisting of allopregnanolone, allotetrahydrodeoxycorticosterone, ganaxolone, alphaxolone, alphadolone, hydroxydione, minaxolone, and Althesin. In some embodiments, the neurosteroid is allopregnanolone. In some embodiments, the steroid is formulated in a cyclodextrin. In various embodiments, the steroid is formulated in hydroxypropyl-beta-cyclodextrin or sulfobutylether-beta-cyclodextrin sodium salt. In some embodiments, the subject is experiencing aura. In some embodiments, the subject has been warned of an impending seizure. In some embodiments, the subject is experiencing a seizure. In some embodiments, the subject has status epilepticus. In some embodiments, the subject has myoclonic epilepsy. In some embodiments, the subject suffers from seizure clusters. In some embodiments, the seizure is a tonic seizure. In some embodiments, the seizure is a clonic seizure. In some embodiments, the subject is a human. In some embodiments, the steroid is administered intramuscularly, intravenously or subcutaneously. In some embodiments, the methods entail treating, reducing, and/or mitigating symptoms associated with and/or caused by epilepsy by intramuscularly (i.m.), subcutaneously (s.c.) or intravenously (i.v.) administering allopregnanolone formulated in a sulfobutylether-beta-cyclodextrin sodium salt. In some embodiments, the epilepsy is status epilepticus. In some embodiments, the steroid or neurosteroid (e.g., allopregnanolone) is administered at a dose in the range of about 0.25 mg/kg to about 15 mg/kg, e.g., about 0.25, 0.5, 1, 2, 3, 4, 5, 6, 7, 8, 9, 10, 11, 12, 13, 14, 15 mg/kg. In some embodiments, the steroid or neurosteroid (e.g., allopregnanolone) is self-administered by the subject. In some embodiments, the steroid or neurosteroid (e.g., allopregnanolone) is administered by a caregiver who is not the subject. In a further aspect, compositions comprising or consisting essentially of a

steroid and a cyclodextrin are provided. In some embodiments, the steroid is a neurosteroid. In some embodiments, the neurosteroid is selected from the group consisting of allopregnanolone, allotetrahydrodeoxycorticosterone, ganaxolone, alphaxolone, alphadolone, hydroxydione, minaxolone, and Althesin. In some embodiments, the steroid is allopregnanolone. In some embodiments, the cyclodextrin is hydroxypropyl-beta-cyclodextrin, sulfobutylether-beta-cyclodextrin sodium salt, or mixture thereof. In some embodiments, the composition comprises allopregnanolone and sulfobutylether-beta-cyclodextrin sodium salt.

In some embodiments, the steroid or neurosteroid (e.g., allopregnanolone) is administered or formulated for administration via an inhaler. In some embodiments, the steroid or neurosteroid (e.g., allopregnanolone) is nebulized or aerosolized. In some embodiments, the steroid or neurosteroid (e.g., allopregnanolone) is nebulized or aerosolized without heating. In some embodiments, the nebulized or aerosolized steroid or neurosteroid (e.g., allopregnanolone) particles have a mass median aerodynamic diameter (“MMAD”) of about 5 μm or smaller. In some embodiments, the nebulized or aerosolized steroid or neurosteroid (e.g., allopregnanolone) particles have a mass median aerodynamic diameter (“MMAD”) of about 2-3 μm. In some embodiments, the steroid or neurosteroid (e.g., allopregnanolone) is delivered to the distal alveoli.

### DEFINITIONS

As used herein, “administering” refers to local and systemic administration, e.g., including enteral, parenteral, pulmonary, and topical/transdermal administration. Routes of administration for steroid or neurosteroids (e.g., allopregnanolone) that find use in the methods described herein include, e.g., oral (per os (P.O.)) administration, nasal, inhalation or intrapulmonary administration, administration as a suppository, topical contact, transdermal delivery (e.g., via a transdermal patch), intrathecal (IT) administration, intravenous (“iv”) administration, intraperitoneal (“ip”) administration, intramuscular (“im”) administration, or subcutaneous (“sc”) administration, or the implantation of a slow-release device e.g., a mini-osmotic pump, a depot formulation, etc., to a subject. Administration can be by any route including parenteral and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, ionophoretic and intracranial. Other modes of delivery include, but are not limited to, the use of liposomal formulations, intravenous infusion, transdermal patches, etc.

The terms “systemic administration” and “systemically administered” refer to a method of administering a compound or composition to a mammal so that the compound or composition is delivered to sites in the body, including the targeted site of pharmaceutical action, via the circulatory system. Systemic administration includes, but is not limited to, oral, intranasal, rectal and parenteral (e.g., other than through the alimentary tract, such as intramuscular, intravenous, intra-arterial, transdermal and subcutaneous) administration.

The term “co-administration” refers to the presence of both active agents in the blood at the same time. Active agents that are co-administered can be delivered concurrently (i.e., at the same time) or sequentially.

The phrase “cause to be administered” refers to the actions taken by a medical professional (e.g., a physician), or a person controlling medical care of a subject, that control and/or permit the administration of the steroid or neurosteroid (e.g., allopregnanolone) to the subject. Causing to be administered can involve diagnosis and/or determination of an appropriate therapeutic or prophylactic regimen, and/or prescribing particular steroid or neurosteroid (e.g., allopregnanolone) for a subject. Such prescribing can include, for example, drafting a prescription form, annotating a medical record, and the like.

The term “effective amount” or “pharmaceutically effective amount” refer to the amount and/or dosage, and/or dosage regime of one or more steroid or neurosteroid (e.g., allopregnanolone) necessary to bring about the desired result e.g., an amount sufficient prevent, abort or terminate a seizure.

As used herein, the terms “treating” and “treatment” refer to delaying the onset of, retarding or reversing the progress of, reducing the severity of, or alleviating or preventing either the disease or condition to which the term applies, or one or more symptoms of such disease or condition.

The terms “reduce,” “inhibit,” “relieve,” “alleviate” refer to the detectable decrease in the frequency, severity and/or duration of seizures. A reduction in the frequency, severity and/or duration of seizures can be measured by self-assessment (e.g., by reporting of the patient) or by a trained clinical observer. Determination of a reduction of the frequency, severity and/or duration of seizures can be made by comparing patient status before and after treatment.

The term “mitigating” refers to reduction or elimination of one or more symptoms of that pathology or disease, and/or a reduction in the rate or delay of onset or severity of one or more symptoms of that pathology or disease (e.g., seizures), and/or the prevention of that pathology or disease.

As used herein, the phrase “consisting essentially of” refers to the genera or species of active pharmaceutical agents (e.g., neurosteroid, e.g., allopregnanolone) and excipient (e.g., hydroxypropyl-beta-cyclodextrin or Captisol (sulfobutylether-beta-cyclodextrin sodium salt)) included in a method or composition. In various embodiments, other unmentioned or unrecited active ingredients and inactive are expressly excluded. In various embodiments, additives (e.g., surfactants, acids (organic or fatty), alcohols, esters, co-solvents, solubilizers, lipids, polymers, glycols) are expressly excluded.

The terms “subject,” “individual,” and “patient” interchangeably refer to a mammal, preferably a human or a non-human primate, but also domesticated mammals (e.g., canine or feline), laboratory mammals (e.g., mouse, rat, rabbit, hamster, guinea pig) and agricultural mammals (e.g., equine, bovine, porcine, ovine). In various embodiments, the subject can be a human (e.g., adult male, adult female, adolescent male, adolescent female, male child, female child) under the care of a physician or other healthworker in a hospital, psychiatric care facility, as an outpatient, or other clinical context. In certain embodiments the subject may not be under the care or prescription of a physician or other healthworker.

The term “neuroactive steroid” or “neurosteroid” refers to steroid compounds that rapidly alter neuronal excitability through interaction with neurotransmitter-gated ion channels. Neurosteroids act as allosteric modulators of neurotransmitter receptors, such as GABAA, NMDA, and sigma receptors. Neurosteroids find use as sedatives for the purpose of general anaesthesia for carrying out surgical procedures, and in the treatment of epilepsy and traumatic brain injury. Illustrative neurosteroids include, e.g., allopregnanolone, Ganaxolone, alphaxolone, alphadolone, hydroxydione, minaxolone, and Althesin (a mixture of alphaxolone and alphadolone).

### BRIEF DESCRIPTION OF THE DRAWINGS

FIG. 1 illustrates a time course for protection by allopregnanolone (5α,3α-P) administered i.v. at a doses of 1.5 and 0.5 mg/kg respectively in the 6-Hz electrical-stimulation (32 mA, 3 s) model. The interval between the steroid injection and the electrical stimulus is plotted on the abscissa and the percentage of animals protected against seizures is plotted on the ordinate. Each point represents eight mice.

FIG. 2 illustrates a time course for protection by allopregnanolone (5α,3α-P) administered i.m. at a doses of 6, 3, 1.5 mg/kg in the 6-Hz electrical stimulation (32 mA, 3 s) model. The interval between the steroid injection and the electrical stimulus is plotted on the abscissa and the percentage of animals protected against seizures is plotted on the ordinate. Each point represents at least eight mice.

FIG. 3 illustrates a time course for protection by allopregnanolone (5α,3α-P) administered s.c. at a doses of 6 and 1.5 mg/kg in the 6-Hz electricalstimulation (32 mA, 3 s) model. The interval between the steroid injection and the electrical stimulus is plotted on the abscissa and the percentage of animals protected against seizures is plotted on the ordinate. Each point represents eight mice.

FIG. 4 illustrates a time course for protection by allopregnanolone (5α,3α-P) administered p.o. at a dose of 300 and 200 mg/kg (double volume of 150 mg/kg and 100 mg/kg suspended/diluted in Canola oil) in the 6-Hz electrical-stimulation (32 mA, 3 s) model. The interval between the steroid injection and the electrical stimulus is plotted on the abscissa and the percentage of animals protected against seizures is plotted on the ordinate. Each point represents at seven to eight mice.

FIG. 5 illustrates the effect of i.v. administration of allopregnanolone (5α,3α-P) (0.1-1.5 mg/kg) on the onset of myoclonic jerk, generalized clonus, and tonic extension in response to PTZ (80 mg/kg, i.p.) injection in mice. 5α,3α-P was administered i.v. 1 min before PTZ injection. Bars indicate mean S.E.M. of values from eight mice. p<0.05 compared with vehicle control group (ANOVA followed by Dunnett's test).

FIG. 6 illustrates the effect of i.v. administration of allopregnanolone (5α,3α-P) (0.1-1.5 mg/kg) on the onset of myoclonic jerk, generalized clonus, and tonic extension in response to PTZ (80 mg/kg, i.p.) injection in mice. 5α,3α-P was administered i.v. 2 min before PTZ injection. Bars indicate mean S.E.M. of values from eight mice. p<0.05 compared with vehicle control group (ANOVA followed by Dunnett's test).

FIG. 7 illustrates the effect of i.v. administration of allopregnanolone (5α,3α-P) (0.25-1.5 mg/kg) on the onset of myoclonic jerk, generalized clonus, and tonic extension in response to PTZ (80 mg/kg, i.p.) injection in mice. 5α,3α-P was administered i.v. 30 min before PTZ injection. Bars indicate mean S.E.M. of values from eight mice. p<0.05 compared with vehicle control group (ANOVA followed by Dunnett's test).

FIG. 8 illustrates the effect of i.m. administration of allopregnanolone (5α,3α-P) (0.25-1.5 mg/kg) on the onset of myoclonic jerk, generalized clonus, and tonic extension in response to PTZ (80 mg/kg, i.p.) injection in mice. 5α,3α-P was administered i.m. 2 min before PTZ injection. Bars indicate mean S.E.M. of values from at least seven mice. p<0.05 compared with vehicle control group (ANOVA followed by Dunnett's test).

FIG. 9 illustrates the effect of i.m. administration of allopregnanolone (5α,3α-P) (0.25-1.5 mg/kg) on the onset of myoclonic jerk, generalized clonus, and tonic extension in response to PTZ (80 mg/kg, i.p.) injection in mice. 5α,3α-P was administered i.m. 30 min before PTZ injection. Bars indicate mean S.E.M. of values from eight mice. p<0.05 compared with vehicle control group (ANOVA followed by Dunnett's test).

FIG. 10 illustrates a Time -concentration profile for plasma allopregnanolone (5α,3α-P) after single i.v. injection in rats. Rats bearing indwelling jugular catheters received single i.v. injection of 5α,3α-P or vehicle and serial blood samples were withdrawn at 1, 2 , 10,15, 30, 60 and 120 min after injection. Plasma was assayed for 5α,3α-P by LC-MS. Each point represents at least 4 animals.

### DETAILED DESCRIPTION

### 1. Introduction

Treatment of status epilepicus requires rapid administration of anti-seizure agents, which are typically delivered either by the intravenous (IV) or intramuscular (IM) routes. Allopregnanolone (3α-hydroxy-5α-pregnan-20-one; 5α,3α-P), an endogenous progesterone-derived steroid that is a positive allosteric modulator of GABA_{A} receptors, is a powerful anti-seizure agent with potential in the treatment of status epilepticus. The present study determines and demonstrates the dosing of allopregnanolone to protect against seizures when delivered intravenously (i.v.), intramuscularly (i.m.), subcutaneously (s.c.) or orally (p.o.).

### 2. Subjects Who Can Benefit

In various embodiments, the subject has a condition that can be treated or mitigated by administration of a neurosteroid, e.g., allopregnanolone. Allopregnanolone has many medical uses, including the treatment, reduction, and/or mitigation of symptoms associated with and/or caused by traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, and smoking cessation. The subject may or may not be exhibiting symptoms.

Accordingly, the invention also contemplates methods of treating, reducing, and/or mitigating symptoms associated with and/or caused by traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, and smoking cessation by administration of a steroid or neurosteroid (e.g., allopregnanolone) dissolved or suspended in a vehicle suitable for systemic administration (e.g., intramuscular, intravenous, subcutaneous), as described herein.

In some embodiments, the subject has epilepsy, has a history of suffering from epileptic seizures or is suffering from epileptic seizures. In various embodiments, the patient may be experiencing an electrographic or behavioral seizure or may be experiencing a seizure aura, which itself is a localized seizure that may spread and become a full blown behavioral seizure. For example, the subject may be experiencing aura that alerts of the impending onset of a seizure or seizure cluster.

Alternatively, the subject may be using a seizure prediction device that alerts of the impending onset of a seizure or seizure cluster. Implantable seizure prediction devices are known in the art and described, e.g., in D'Alessandro, et al., IEEE TRANSACTIONS ON BIOMEDICAL ENGINEERING, VOL. 50, NO. 5, MAY 2003, and U.S. Patent Publication Nos. 2010/0198098, 2010/0168603, 2009/0062682, and 2008/0243022.

The subject may have a personal or familial history of any of the epileptic conditions described herein. The subject may have been diagnosed as having any of the epileptic conditions described herein. In some embodiments, the subject has or is at risk of suffering a myoclonic seizure or myoclonic epilepsy, e.g., juvenile myoclonic epilepsy. The PTZ seizure model demonstrated herein is predictive of utility and/or activity in counteracting myoclonic seizures or myoclonic epilepsy in humans.

In various embodiments, the subject may be at risk of exposure to or may have been exposed to a nerve agent or a pesticide that can cause seizures. Illustrative nerve agents that can cause seizures include, e.g., organophosphorus nerve agents, e.g., tabun, sarin, soman, GF, VR and/or VX. Illustrative pesticides that can cause seizures include, e.g., organophosphate pesticides (e.g., Acephate (Orthene), Azinphos-methyl (Gusathion, Guthion), Bensulide (Betasan, Lescosan), Bomyl (Swat), Bromophos (Nexion), Bromophos-ethyl (Nexagan), Cadusafos (Apache, Ebufos, Rugby), Carbophenothion (Trithion), Chlorethoxyfos (Fortress), Chlorfenvinphos (Apachlor, Birlane), Chlormephos (Dotan), Chlorphoxim (Baythion-C), Chlorpyrifos (Brodan, Dursban, Lorsban),

Chlorthiophos (Celathion), Coumaphos (Asuntol, Co-Ral), Crotoxyphos (Ciodrin, Cypona), Crufomate (Ruelene), Cyanofenphos (Surecide), Cyanophos (Cyanox), Cythioate (Cyflee, Proban), DEF (De-Green), E-Z-Off D), Demeton (Systox), Demeton-S-methyl (Duratox, Metasystoxl), Dialifor (Torak), Diazinon, Dichlorofenthion, (VC-13 Nemacide), Dichlorvos (DDVP, Vapona), Dicrotophos (Bidrin), Dimefos (Hanane, Pestox XIV), Dimethoate (Cygon, DeFend), Dioxathion (Delnav), Disulfoton (Disyston), Ditalimfos, Edifenphos, Endothion, EPBP (S-seven), EPN, Ethion (Ethanox), Ethoprop (Mocap), Ethyl parathion (E605, Parathion, thiophos), Etrimfos (Ekamet), Famphur (Bash, Bo-Ana, Famfos), Fenamiphos (Nemacur), Fenitrothion (Accothion, Agrothion, Sumithion), Fenophosphon (Agritox, trichloronate), Fensulfothion (Dasanit), Fenthion (Baytex, Entex, Tiguvon), Fonofos (Dyfonate, N-2790), Formothion (Anthio), Fosthietan (Nem-A-Tak), Heptenophos (Hostaquick), Hiometon (Ekatin), Hosalone (Zolone), IBP (Kitazin), Iodofenphos (Nuvanol-N), Isazofos (Brace, Miral, Triumph), Isofenphos (Amaze, Oftanol), Isoxathion (E-48, Karphos), Leptophos (Phosvel), Malathion (Cythion), Mephosfolan (Cytrolane), Merphos (Easy Off-D, Folex), Methamidophos (Monitor), Methidathion (Supracide, Ultracide), Methyl parathion (E601, Penncap-M), Methyl trithion, Mevinphos (Duraphos, Phosdrin), Mipafox (Isopestox, Pestox XV), Monocrotophos (Azodrin), Naled (Dibrome), Oxydemeton-methyl (Metasystox-R), Oxydeprofos (Metasystox-S), Phencapton (G 28029), Phenthoate (Dimephenthoate, Phenthoate), Phorate (Rampart, Thimet), Phosalone (Azofene, Zolone), Phosfolan (Cylan, Cyolane), Phosmet (Imidan, Prolate), Phosphamidon (Dimecron), Phostebupirim (Aztec), Phoxim (Baythion), Pirimiphos-ethyl (Primicid), Pirimiphos-methyl (Actellic), Profenofos (Curacron), Propetamphos (Safrotin), Propyl thiopyrophosphate (Aspon), Prothoate (Fac), Pyrazophos (Afugan, Curamil), Pyridaphenthion (Ofunack), Quinalphos (Bayrusil), Ronnel (Fenchlorphos, Korlan), Schradan (OMPA), Sulfotep (Bladafum, Dithione, Thiotepp), Sulprofos (Bolstar, Helothion), Temephos (Abate, Abathion), Terbufos (Contraven, Counter), Tetrachlorvinphos (Gardona, Rabon), Tetraethyl pyrophosphate (TEPP), Triazophos (Hostathion), and Trichlorfon (Dipterex, Dylox, Neguvon, Proxol).

### 3. Steroids

The compositions generally comprise or consist essentially of a steroid, e.g., a neurosteroid, suspended or dissolved in vehicle appropriate for systemic administration, e.g., a cyclodextin, e.g., hydroxypropyl-beta-cyclodextrin or sulfobutylether-beta-cyclodextrin sodium salt, or mixtures thereof.

In various embodiments the neurosteroid is allopregnanolone (ALP). Allopregnanolone, also known as 3α-hydroxy-5α-pregnan-20-one or 3α,5α-tetrahydroprogesterone, IUPAC name 1-(3-Hydroxy-10,13-dimethyl-2,3,4,5,6,7,8,9,11,12,14,15,16,17-tetradecahydro-1H-cyclopenta[a]phenanthren-17-yl)ethanone, and referenced as CAS number 516-54-1, is a prototypic neurosteroid present in the blood and also the brain. It is a metabolite of progesterone and modulator of GABA_{A} receptors. While allopregnanolone, like other GABA_{A} receptor active neurosteroids such as allotetrahydrodeoxycorticosterone (3α,21-dihydroxy-5α-pregnan-20-one; THDOC), positively modulates all GABA_{A} receptor isoforms, those isoforms containing 8-subunits exhibit greater magnitude potentiation. Allopregnanolone has pharmacological properties similar to other positive modulators of GABA_{A} receptors, including anxiolytic and anticonvulsant activity. Allopregnanolone is neuroprotective in many animal models of neurodegenerative conditions, including, e.g., Alzheimer's disease (Wang et al., Proc Natl Acad Sci USA. 2010 Apr 6;107(14):6498-503), cerebral edema (Limmroth et al., Br J Pharmacol. 1996 Jan; 117(1):99-104) and traumatic brain injury (He et al., Restor Neurol Neurosci. 2004;22(1): 19-31; and He, et al., Exp Neurol. 2004 Oct; 189(2):404-12), Mood disorders (Robichaud and Debonnel, Int J Neuropsychopharmacol. 2006 Apr;9(2): 191-200), Niemann-Pick type C disease (Griffin et al., Nat Med. 2004 Jul; 10(7):704-11) and acts as an anticonvulsant against chemically induced seizures, including the pentylenetetrazol (PTZ) model (Kokate et al., J Pharmacol Exp Ther. 1994 Sep;270(3):1223-9). The chemical structure of allopregnanolone is depicted below in Formula I:

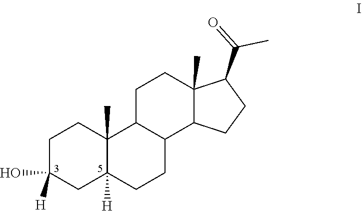 [SMILES: CC(=O)[C@H]1CCC2C3CC[C@H]4C[C@H](O)CC[C@]4(C)C3CC[C@@]21C]

In various embodiments, the compositions comprise a sulfate, salt, hemisuccinate, nitrosylated, derivative or congener of allopregnanolone.

Other neurosteroids that can be formulated in vehicle suitable for systemic administration, include without limitation allotetrahydrodeoxycorticosterone (3α,21-dihydroxy-5α-pregnan-20-one; THDOC), 3α,21-dihydroxy-5b-pregnan-20-one, pregnanolone (3α-hydroxy-5-pregnan-20-one), Ganaxolone (INN, also known as CCD-1042; IUPAC name (3α,5α)-3-hydroxy-5-methylpregnan-20-one; 1-[(3R,5S,8R,9S,10S,13S, 14S, 17S)-3-hydroxy-3,10,13-trimethyl-1,2,4,5,6,7,8,9,11,12,14,15,16,17-tetradecahydrocyclopenta[a]phenanthren-17-yl]ethanone), alphaxolone, alphadolone, hydroxydione, minaxolone, and Althesin (a mixture of alphaxolone, alphadolone, tetrahydrodeoxycorticosterone, pregnenolone, dehydroepiandrosterone (DHEA), 7-substituted benz[e]indene-3-carbonitriles (see, e.g., Hu, et al., J Med Chem. (1993) 36(24):3956-67); 7-(2-hydroxyethyl)benz[e]indene analogues (see, e.g., Han, et al., J Med Chem. (1995) 38(22):4548-56); 3 alpha-hydroxy-5 alpha-pregnan-20-one and 3 alpha-hydroxy-5 beta-pregnan-20-one analogues (see, e.g., Han, et al., J Med Chem. (1996) 39(21):4218-32); enantiomers of dehydroepiandrosterone sulfate, pregnenolone sulfate, and (3alpha,5beta)-3-hydroxypregnan-20-one sulfate (see, e.g., Nilsson, et al., J Med Chem. (1998) 41(14):2604-13); 13,24-cyclo-18,21-dinorcholane analogues (see, e.g., Jiang, et al., J Med Chem. (2003) 46(25):5334-48); N-acylated 17a-aza-D-homosteroid analogues (see, e.g., Covey, et al., J Med Chem. (2000) 43(17):3201-4); 5 beta-methyl-3-ketosteroid analogues (see, e.g., Zeng, et al., J Org Chem. (2000) 65(7):2264-6); 18-norandrostan-17-one analogues (see, e.g., Jiang, et al., J Org Chem. (2000) 65(11):3555-7); (3alpha,5alpha)- and (3alpha,5beta)-3-hydroxypregnan-20-one analogs (see, e.g., Zeng, et al., J Med Chem. (2005) 48(8):3051-9); benz[f]indenes (see, e.g., Scaglione, et al., J Med Chem. (2006) 49(15):4595-605); enantiomers of androgens (see, e.g., Katona, et al., Eur J Med Chem. (2008) 43(1): 107-13); cyclopenta[b]phenanthrenes and cyclopenta[b]anthracenes (see, e.g., Scaglione, et al., J Med Chem. (2008) 51(5):1309-18); 2beta-hydroxygonane derivatives(see, e.g., Wang, et al., Tetrahedron (2007) 63(33):7977-7984); 416-alphaxalone and corresponding 17-carbonitrile analogues (see, e.g., Bandyopadhyaya, et al., Bioorg Med Chem Lett. (2010) 20(22):6680-4); Δ(16) and Δ(17(20)) analogues of Δ(16)-alphaxalone (see, e.g., Stastna, et al., J Med Chem. (2011) 54(11):3926-34); neurosteroid analogs developed by CoCensys (now Purdue Neuroscience) (e.g., CCD-3693, Co2-6749 (a.k.a., GMA-839 and WAY-141839); neurosteroid analogs described in U.S. Pat. No. 7,781,421 and in PCT Patent Publications WO 2008/157460; WO 1993/003732; WO 1993/018053; WO 1994/027608; WO 1995/021617; WO 1996/016076; WO 1996/040043, as well as salts, hemisuccinates, nitrosylated, sulfates and derivatives thereof.

In various embodiments, the steroid or neurosteroid is not a sex hormone. In various embodiments, the steroid or neurosteroid is not progesterone.

As appropriate, the steroid or neurosteroid (e.g., allopregnanolone) may or may not be micronized. As appropriate, the steroid or neurosteroid (e.g., allopregnanolone) may or may not be enclosed in microspheres in suspension in the oil.

### 4. Formulation and Administration

In varying embodiments, the steroid and/or an analog thereof can be administered systemically, e.g., intramuscularly (IM), or depo-IM, subcutaneously (SQ), and depo-SQ), as appropriate or desired. In varying embodiments, the dosage form is selected to facilitate delivery to the brain (e.g., passage through the blood brain barrier). In this context it is noted that the steroids or neurosteroids (e.g., allopregnanolone) described herein can be readily delivered to the brain. Dosage forms known to those of skill in the art are suitable for delivery of the steroid.

Compositions are provided that contain therapeutically effective amounts of the steroid or neurosteroid (e.g., allopregnanolone). The steroids or neurosteroids (e.g., allopregnanolone) are preferably formulated into suitable pharmaceutical preparations such as tablets, capsules, or elixirs for oral administration or in sterile solutions or suspensions for parenteral administration. Typically the steroids or neurosteroids (e.g., allopregnanolone) described above are formulated into pharmaceutical compositions using techniques and procedures well known in the art.

These steroids or neurosteroids (e.g., allopregnanolone) or analogs thereof can be administered in the “native” form or, if desired, in the form of salts, esters, amides, prodrugs, derivatives, and the like, provided the salt, ester, amide, prodrug or derivative is suitable pharmacologically effective, e.g., effective in the present method(s). Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

Methods of formulating such derivatives are known to those of skill in the art. For example, the disulfide salts of a number of delivery agents are described in PCT Publication WO 2000/059863 which is incorporated herein by reference. Similarly, acid salts of therapeutic peptides, peptoids, or other mimetics, and can be prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include, but are not limited to both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, orotic acid, and the like, as well as inorganic acids, e.g., hydrochloric acid, hydrobromic acid, sulfuric acid, nitric acid, phosphoric acid, and the like. An acid addition salt can be reconverted to the free base by treatment with a suitable base. Certain particularly preferred acid addition salts of the active agents herein include halide salts, such as may be prepared using hydrochloric or hydrobromic acids. Conversely, preparation of basic salts of the active agents of this invention are prepared in a similar manner using a pharmaceutically acceptable base such as sodium hydroxide, potassium hydroxide, ammonium hydroxide, calcium hydroxide, trimethylamine, or the like. In certain embodiments basic salts include alkali metal salts, e.g., the sodium salt, and copper salts.

For the preparation of salt forms of basic drugs, the pKa of the counterion is preferably at least about 2 pH lower than the pKa of the drug. Similarly, for the preparation of salt forms of acidic drugs, the pKa of the counterion is preferably at least about 2 pH higher than the pKa of the drug. This permits the counterion to bring the solution's pH to a level lower than the pHmax to reach the salt plateau, at which the solubility of salt prevails over the solubility of free acid or base. The generalized rule of difference in pKa units of the ionizable group in the active pharmaceutical ingredient (API) and in the acid or base is meant to make the proton transfer energetically favorable. When the pKa of the API and counterion are not significantly different, a solid complex may form but may rapidly disproportionate (e.g., break down into the individual entities of drug and counterion) in an aqueous environment.

Preferably, the counterion is a pharmaceutically acceptable counterion. Suitable anionic salt forms include, but are not limited to acetate, benzoate, benzylate, bitartrate, bromide, carbonate, chloride, citrate, edetate, edisylate, estolate, fumarate, gluceptate, gluconate, hydrobromide, hydrochloride, iodide, lactate, lactobionate, malate, maleate, mandelate, mesylate, methyl bromide, methyl sulfate, mucate, napsylate, nitrate, pamoate (embonate), phosphate and diphosphate, salicylate and disalicylate, stearate, succinate, sulfate, tartrate, tosylate, triethiodide, valerate, and the like, while suitable cationic salt forms include, but are not limited to aluminum, benzathine, calcium, ethylene diamine, lysine, magnesium, meglumine, potassium, procaine, sodium, tromethamine, zinc, and the like.

In various embodiments preparation of esters typically involves functionalization of hydroxyl and/or carboxyl groups that are present within the molecular structure of the active agent. In certain embodiments, the esters are typically acyl-substituted derivatives of free alcohol groups, e.g., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine.

Determination of an effective amount for administration in a single dosage is well within the capability of those skilled in the art, especially in light of the detailed disclosure provided herein. Generally, an efficacious or effective amount of the steroid or neurosteroid (e.g., allopregnanolone) is determined by first administering a low dose or small amount of the agent and then incrementally increasing the administered dose or dosages, adding a second or third medication as needed, until a desired effect of is observed in the treated subject with minimal or no toxic side effects. Applicable methods for determining an appropriate dose and dosing schedule for administration of a combination of the present invention are described, for example, in Brunton, et al., Goodman and Gilman's The Pharmacological Basis of Therapeutics, 12th Edition, 2010, McGraw-Hill Professional; in a Physicians' Desk Reference (PDR), 66th Edition, 2012; in Loyd, et al., Remington: The Science and Practice of Pharmacy, 22st Ed., 2012, Pharmaceutical Press; in Martindale: The Complete Drug Reference, Sweetman, 2005, London: Pharmaceutical Press., and in Martindale, Martindale: The Extra Pharmacopoeia, 31st Edition., 1996, Amer Pharmaceutical Assn, each of which are hereby incorporated herein by reference. In various embodiments, the compositions are formulated, e.g., for oral administration, at a dose in the range of about 5 mg/kg to about 250 mg/kg of the steroid or neurosteroid (e.g., allopregnanolone), e.g., about 5 mg/kg, 10 mg/kg, 15 mg/kg, 20 mg/kg, 25 mg/kg, 30 mg/kg, 35 mg/kg, 40 mg/kg, 45 mg/kg, 50 mg/kg, 75 mg/kg, 100 mg/kg, 125 mg/kg, 150 mg/kg, 200 mg/kg, or 250 mg/kg.

About 1 to 1000 mg of a steroid or neurosteroid (e.g., allopregnanolone), or a physiologically acceptable salt or ester is compounded with a physiologically acceptable vehicle, carrier, excipient, binder, preservative, stabilizer, flavor, etc., in a unit dosage form as called for by accepted pharmaceutical practice. The amount of active substance in those compositions or preparations is such that a suitable dosage in the range indicated is obtained. The compositions are preferably formulated in a unit dosage form, each dosage containing from about 1-1000 mg, 2-800 mg, 5-500 mg, 10-400 mg, 50-200 mg, e.g., about 5 mg, 10 mg, 15 mg, 20 mg, 25 mg, 30 mg, 35 mg, 40 mg, 45 mg, 50 mg, 60 mg, 70 mg, 80 mg, 90 mg, 100 mg, 200 mg, 300 mg, 400 mg, 500 mg, 600 mg, 700 mg, 800 mg, 900 mg or 1000 mg of the active ingredient. In varying embodiments, the steroid or neurosteroid (e.g., allopregnanolone) is administered systemically (e.g., intramuscularly, intravenously, subcutaneously) at a dose in the range of about 0.25 mg/kg to about 15 mg/kg, e.g., about 0.25 mg/kg to about 15 mg/kg, e.g., about 0.25, 0.5, 1, 2, 3, 4, 5, 6, 7, 8, 9, 10, 11, 12, 13, 14, 15 mg/kg. The term “unit dosage from” refers to physically discrete units suitable as unitary dosages for human subjects and other mammals, each unit containing a predetermined quantity of active material calculated to produce the desired therapeutic effect, in association with a suitable pharmaceutical excipient.

In varying embodiments, the steroids or neurosteroids (e.g., allopregnanolone) are formulated for intrapulmonary administration. In various embodiments, the steroids or neurosteroids (e.g., allopregnanolone) are formulated for delivery via an inhaler.

In various embodiments, the steroids or neurosteroids (e.g., allopregnanolone) are nebulized. Methods and systems for intrapulmonary delivery of steroids or neurosteroids (e.g., allopregnanolone) are known in the art and find use. Illustrative systems for aerosol delivery of steroids or neurosteroids (e.g., allopregnanolone) by inhalation are described, e.g., in U.S. Pat. Nos. 5,497,763; 5,660,166; 7,060,255; and 7,540,286; and U.S. Patent Publication Nos. 2003/0032638; and 2006/0052428, each of which are hereby incorporated herein by reference in their entirety for all purposes. Preferably, the steroids or neurosteroids (e.g., allopregnanolone) are nebulized without the input of heat.

For administration of the nebulized and/or aerosolized steroids or neurosteroids (e.g., allopregnanolone), the size of the aerosol particulates can be within a range appropriate for intrapulmonary delivery, particularly delivery to the distal alveoli. In various embodiments, the aerosol particulates have a mass median aerodynamic diameter (“MMAD”) of less than about 5 μm, 4 μm, 3 μm, for example, ranging from about 1 μm to about 3 μm, e.g., from about 2 μm to about 3 μm, e.g., ranging from about 0.01 μm to about 0.10 μm. Aerosols characterized by a MMAD ranging from about 1 μm to about 3 μm can deposit on alveoli walls through gravitational settling and can be absorbed into the systemic circulation, while acrosols characterized by a MMAD ranging from about 0.01 μm to 0.10 μm can also be deposited on the alveoli walls through diffusion. Acrosols characterized by a MMAD ranging from about 0.15 μm to about 1 μm are generally exhaled. Thus, in various embodiments, aerosol particulates can have a MMAD ranging from 0.01 μm to about 5 μm, for example, ranging from about 0.05 μm to about 3 μm, for example, ranging from about 1 μm to about 3 μm, for example, ranging from about 0.01 μm to about 0.1 μm. The nebulized and/or aerosolized steroids or neurosteroids (e.g., allopregnanolone) can be delivered to the distal alveoli, allowing for rapid absorption and efficacy.

In various embodiments, the steroids or neurosteroids (e.g., allopregnanolone) is formulated in a solution comprising excipients suitable for aerosolized intrapulmonary delivery. The solution can comprise one or more pharmaceutically acceptable carriers and/or excipients. Pharmaceutically acceptable refers to approved or approvable by a regulatory agency of the Federal or a state government or listed in the U.S Pharmacopoeia or other generally recognized pharmacopoeia for use in animals, and more particularly in humans. Preferably, the solution is buffered such that the solution is in a relatively neutral pH range, for example, a pH in the range of about 4 to 8, for example, a pH in the range of about 5-7. In some embodiments, the steroids or neurosteroids (e.g., allopregnanolone) is formulated in a buffered solution, for example, phosphate-buffered saline.

In various embodiments, the steroids or neurosteroids (e.g., allopregnanolone) is prepared as a concentrated aqueous solution. Ordinary metered dose liquid inhalers have poor efficiency for the delivery to the deep lung because the particle size is not sufficiently small (Kim et al., 1985 Am Rev Resp Dis 132:137-142; and Farr et al., 1995 Thorax 50:639-644). These systems are therefore used mostly for local delivery of drugs to the pulmonary airways. In addition, metered doses inhalers may not be able to deliver sufficient volumes of even a concentrated steroids or neurosteroids (e.g., allopregnanolone) solution to produce the desired rapid antiseizure effect. Accordingly, in various embodiments, a metered doses inhaler is not used for delivery of the steroids or neurosteroids (e.g., allopregnanolone). In one embodiment a nebulization system with the capability of delivering <5 μm particles (e.g., the PARI LC Star, which has a high efficiency, 78% respirable fraction 0.1-5 μm. see, e.g., pari.com) is used for intrapulmonary administration. Electronic nebulizers which employ a vibrating mesh or aperture plate to generate an aerosol with the required particle size can deliver sufficient quantities rapidly and find use (See, e.g., Knoch and Keller, 2005 Expert Opin Drug Deliv 2: 377-390). Also, custom-designed hand-held, electronic nebulizers can be made and find use.

Aerosolized delivery of steroids or neurosteroids (e.g., allopregnanolone) allows for reduced dosing to achieve desired efficacy, e.g., in comparison to intravenous or intranasal delivery. Appropriate dosing will depend on the size and health of the patient and can be readily determined by a trained clinician. Initial doses are low and then can be incrementally increased until the desired therapeutic effect is achieved with little or no adverse side effects. In various embodiments, the steroids or neurosteroids (e.g., allopregnanolone) are administered via the intrapulmonary route at a dose that is about 10%, 15%, 25%, 50% or 75% of established doses for their administration via other routes (e.g., via oral, intravenous or intranasal administration). In some embodiments, the steroids or neurosteroids (e.g., allopregnanolone) are administered via the intrapulmonary route at a dose in the range of about 0.05 mg/kg to about 1.0 mg/kg, for example, about 0.2 mg/kg to about 0.8 mg/kg, for example, about 0.05 mg/kg, 0.08 mg/kg, 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, or 1.0 mg/kg. In some embodiments, the steroids or neurosteroids (e.g., allopregnanolone) are administered via the intrapulmonary route at a dose in the range of about 10 μg/kg to about 80 μg/kg, for example, about 20 μg/kg to about 60 μg/kg, for example, about 25 μg/kg to about 50 μg/kg, for example, about 10 μg/kg, 15 μg/kg, 20 μg/kg, 25 μg/kg, 30 μg/kg, 35 μg/kg, 40 μg/kg, 45 μg/kg, 50 μg/kg, 60 μg/kg, 70 μg/kg, or 80 μg/kg. In some embodiments, the steroids or neurosteroids (e.g., allopregnanolone) are administered via the intrapulmonary route at a dose in the range of about 0.3 μg/kg to about 3.0 μg/kg.

To prepare compositions, the steroid or neurosteroid (e.g., allopregnanolone) is mixed with a suitable pharmaceutically acceptable carrier. Upon mixing or addition of the compound(s), the resulting mixture may be a solution, suspension, emulsion, or the like. Liposomal suspensions may also be suitable as pharmaceutically acceptable carriers. These may be prepared according to methods known to those skilled in the art. The form of the resulting mixture depends upon a number of factors, including the intended mode of administration and the solubility of the steroid or neurosteroid (e.g., allopregnanolone) in the selected carrier or vehicle. The effective concentration is sufficient for lessening or ameliorating at least one symptom of the disease, disorder, or condition treated and may be empirically determined.

Pharmaceutical carriers or vehicles suitable for administration of the steroids or neurosteroids (e.g., allopregnanolone) provided herein include any such carriers known to those skilled in the art to be suitable for the particular mode of administration (e.g., cyclodextrins). In addition, the active materials can also be mixed with other active materials that do not impair the desired action, or with materials that supplement the desired action, or have another action. The steroids or neurosteroids (e.g., allopregnanolone) may be formulated as the sole pharmaceutically active ingredient in the composition or may be combined with other active ingredients.

Where the steroids or neurosteroids (e.g., allopregnanolone) exhibit insufficient solubility, methods for solubilizing may be used. Such methods are known and include, but are not limited to, using cosolvents such as dimethylsulfoxide (DMSO), using surfactants such as Tween™, and dissolution in aqueous sodium bicarbonate. Derivatives of the steroids or neurosteroids (e.g., allopregnanolone), such as salts or prodrugs may also be used in formulating effective pharmaceutical compositions.

The concentration of the steroid or neurosteroid (e.g., allopregnanolone) is effective for delivery of an amount upon administration that lessens or ameliorates at least one symptom of the disorder for which the compound is administered and/or that is effective in a prophylactic context. Typically, the compositions are formulated for single dosage (e.g., daily) administration.

The steroids or neurosteroids (e.g., allopregnanolone) may be prepared with carriers that protect them against rapid elimination from the body, such as time-release formulations or coatings. Such carriers include controlled release formulations, such as, but not limited to, microencapsulated delivery systems. The active steroid or neurosteroid (e.g., allopregnanolone) is included in the pharmaceutically acceptable carrier in an amount sufficient to exert a therapeutically useful effect in the absence of undesirable side effects on the patient treated. The therapeutically effective concentration may be determined empirically by testing the steroids or neurosteroids (e.g., allopregnanolone) in known in vitro and in vivo model systems for the treated disorder. A therapeutically or prophylactically effective dose can be determined by first administering a low dose, and then incrementally increasing until a dose is reached that achieves the desired effect with minimal or no undesired side effects.

In various embodiments, the steroids or neurosteroids (e.g., allopregnanolone) and/or analogs thereof can be enclosed in multiple or single dose containers. The enclosed compounds and compositions can be provided in kits, for example, including component parts that can be assembled for use. For example, a compound inhibitor in lyophilized form and a suitable diluent may be provided as separated components for combination prior to use. A kit may include a compound inhibitor and a second therapeutic agent for co-administration. The inhibitor and second therapeutic agent may be provided as separate component parts. A kit may include a plurality of containers, each container holding one or more unit dose of the compounds. The containers are preferably adapted for the desired mode of administration, including, but not limited to depot products, pre-filled syringes, ampules, vials, and the like for parenteral administration

The concentration and/or amount of steroid or neurosteroid (e.g., allopregnanolone) in the drug composition will depend on absorption, inactivation, and excretion rates of the steroid or neurosteroid (e.g., allopregnanolone), the dosage schedule, and amount administered as well as other factors known to those of skill in the art.

The active ingredient may be administered at once, or may be divided into a number of smaller doses to be administered at intervals of time. It is understood that the precise dosage and duration of treatment is a function of the disease being treated and may be determined empirically using known testing protocols or by extrapolation from in vivo or in vitro test data. It is to be noted that concentrations and dosage values may also vary with the severity of the condition to be alleviated. It is to be further understood that for any particular subject, specific dosage regimens should be adjusted over time according to the individual need and the professional judgment of the person administering or supervising the administration of the compositions, and that the concentration ranges set forth herein are exemplary only and are not intended to limit the scope or practice of the claimed compositions.

### 5. Monitoring Efficacy

In various embodiments, administration of a steroid or neurosteroid (e.g., allopregnanolone) to a subject results in the prevention or mitigation of one or more symptoms of the disease condition being treated (e.g., traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, and smoking cessation). Symptoms of disease can be compared before and after administration of a steroid or neurosteroid (e.g., allopregnanolone) to the subject. Administration of the steroid or neurosteroid (e.g., allopregnanolone) to the subject is considered to be effective if the symptoms no longer occur after administration (e.g., seizures), or if the symptoms are reduced, alleviated and/or mitigated after administration.

In various embodiments, administration of a steroid or neurosteroid (e.g., allopregnanolone) to a subject results in the prevention of the occurrence of an impending seizure and/or the termination or abortion of a seizure in progress.

In various embodiments, efficacy can be monitored by the subject. For example, in a subject experiencing aura or receiving a warning from a seizure prediction device, the subject can self-administer a dose of the steroid or neurosteroid (e.g., allopregnanolone). If the steroid or neurosteroid (e.g., allopregnanolone) is administered in an efficacious amount, the sensation of aura should subside and/or the seizure prediction device should no longer predict the imminent occurrence of an impending seizure. If the sensation of aura does not subside and/or the seizure prediction device continues to predict an impending seizure, a second dose of steroid or neurosteroid (e.g., allopregnanolone) can be administered.

In other embodiments, the efficacy is monitored by a caregiver. For example, in a subject experiencing the onset of a seizure or in situations where a seizure has commenced, the subject may require administration of the steroid or neurosteroid (e.g., allopregnanolone) by a caregiver. If the steroid or neurosteroid (e.g., allopregnanolone) is administered in an efficacious amount, the seizure, along with the subject's symptoms of the seizure, should terminate or abort. If the seizure does not terminate, a second dose of the steroid or neurosteroid (e.g., allopregnanolone) can be administered.

### EXAMPLES

The following examples are offered to illustrate, but not to limit the claimed invention.

### Example 1

### Anticonvulsant Activity of Intravenous and Intramuscular Allopregnanolone

### Rationale:

Treatment of status epilepicus requires rapid administration of antiseizure agents, which are typically delivered either by the intravenous (i.v.) or intramuscular (i.m.) routes. Allopregnanolone (3α-hydroxy-5α-pregnan-20-one; 5α,3α-P), an endogenous progesterone-derived steroid that is a positive allosteric modulator of GABA_{A} receptors, is a powerful antiseizure agent with potential in the treatment of status epilepticus. The objective of this study was to determine the dosing of allopregnanolone to protect against seizures when delivered i.v. and i.m.

### Methods:

The mouse 6 Hz and pentylenetetrazol seizure models were used. Solutions of 5α,3α-P were made in 6% (0.5 and 1.5 mg/ml) sulfobutylether-β-cyclodextrin sodium salt (Captisol®) in 0.9% saline. The solutions were injected i.v. or i.m. (1, 2 and 30 min or 2 and 30 min, respectively) prior to administration of the 6 Hz electrical stimulus or PTZ (80 mg/kg, i.p.). In case of the PTZ model, animals were observed for 30 min and times to myoclonic jerks and clonic and tonic seizures were recorded. Anticonvulsant activity was assessed by the delay in onset of seizure signs. Allopregnanolone plasma levels in rats were determined by LC-MS.

### Results:

5α,3α-P exhibited protective activity in the 6 Hz test 1-15 min after i.v. infusion (1.5 mg/kg) but was inactive at 30 min. In contrast, with i.m. administration (3 mg/kg) the onset of protective activity was slower (within 2 min) and lasted <2 h. At a dose of 0.1 mg/kg i.v. 5α,3α-P failed to significantly delay seizure onset in the PTZ model at all pretreatment times (1, 2 and 30 min) whereas a dose of 0.5 mg/kg administered 1 min before PTZ caused a marked delay for myoclonic jerks and clonic seizures and in 62.5% of animals prevented tonic seizures and mortality that invariably accompanies tonic seizures. When injected 2 min before PTZ 5α,3α-P (0.5 mg/kg) caused a similar increase in time to onset of seizures signs and prevented tonic seizures in 25% of animals.

5α,3α-P at a dose of 1.5 mg/kg completely prevented tonic seizures and mortality when injected i.v. 1 and 2 min before PTZ. When injected i.m. 2 min before PTZ, 0.25, 0.5 and 1.5 mg/kg 5α,3α-P protected 0%, 50% and 100%, respectively, of animals from tonic seizures. 5α,3α-P at the dose of 1.5 mg/kg i.m. provided significant protection against tonic seizures when injected 30 min before PTZ; the same dose injected i.v. 30 min before PTZ was inactive. In rats, an i.v. bolus dose of 0.5 and 1.0 mg/kg 5α,3α-P caused mean peak plasma levels (2 min) of 337 and 746 ng/ml, respectively; for both doses, the pooled mean two component halftimes were 2 and 22 min.

### Conclusions:

Our results demonstrate that i.v. 5α,3α-P provides very rapid but transitory anticonvulsant activity. When injected i.m., 5α,3α-P acts comparably quickly and has a longer duration of action. Parenteral 5α,3α-P may be useful for the acute treatment of seizures.

### Detailed Methods

Animals. Male NIH Swiss mice (22-30 g) served as subjects, and all procedures used in these studies were conducted in accordance with the University of California, Davis, Institutional Animal Care and Use Committee the Animal Care and Use policies in strict compliance with the Guide for the Care and Use of Laboratory Animals of the National Research Council (National Academy Press, Washington, DC; on the internet at nap.edu/readingroom/books/labrats/).

Test substances and drug administration. Allopregnanolone (3α-hydroxy-5α-pregnan-20-one; 5α,3α-P) was synthesized by a SAFC Pharma Inc, Madison, WI, USA and Captisol (sulfobutylether-beta-cyclodextrin sodium salt) was provided by Ligand Pharmaceuticals, Inc. La Jolla, CA, USA. Solutions of 5α,3α-P were made in 6% (0.5 and 1.5 mg/ml) or 24% (6 mg/kg) sulfobutylether-β-cyclodextrin sodium salt (Captisol®) in 0.9% saline. The volumes used for all injections were 10-20 ml/kg of body weight. In order to establish time courses for protection by 5α,3α-P in the 6 -Hz electrical-stimulation (32 mA, 3 s) model, 5α,3α-P (0.5-6 mg/kg) was administered intravenously (i.v.), intramuscularly (i.m.), subcutaneously (s.c.) or orally (p.o.) before electrical stimulation. In the PTZ seizure test, 5α,3α-P or vehicle were administered i.v. or i.m. 1, 2 or 30 min before PTZ.

Seizures Models 6 -Hz seizure test (Kaminski, et al., Epilepsia (2004) 45:1-4): 3-s corneal stimulation (200-μs duration, 32-mA monopolar rectangular pulses at 6 Hz) was delivered by a constant-current device (ECT Unit 5780; Ugo Basile, Comerio, Italy). After the stimulation, the animals exhibited a “stunned” posture associated with rearing and automatic movements that lasted from 60 to 120 s in untreated animals. The experimental end point was protection against the seizure: an animal was considered to be protected if it resumed its normal exploratory behavior within 10 s of stimulation.

Pentylenetetrazol seizure test (Kokate, et al., J Pharmacol Exp Ther (1994) 270:1223-9): mice were injected intraperitoneally with PTZ (80 mg/kg) and were observed for a 30-min period. The time of onset of myoclonic jerks, clonus and tonic extension was recorded.

Surgery and blood collection. Male rats were implanted with indwelling jugular catheters as described (Baumann, et al., J Neurosci. (1998) 18: 9069-77). Animals were allowed to recover for at least one week. Experiments were carried out while the animal resided in its home cage. Rats received i.v. injection of vehicle or 5α,3α-P and serial blood samples were withdrawn into chilled tubes at 1, 2, 10,15, 30, 60 and 120 min after i.v. injection. 5α,3α-P and D4- 5α,3α-P (internal standard) were extracted with SPE method from rat's plasma. The extracted 5α,3α-P and D4-5α,3α-P were quantified with ultra-performance liquid chromatography (UPLC)/Atmospheric-pressure chemical ionization (APCI)/ tandem mass spectrometry (MS/MS).

Data analysis. Results are expressed as mean±S.E.M.; the significance of the difference in the responses of treatment groups with respect to control is based on one-way analysis of variance (ANOVA) followed by specific post hoc comparisons using Dunnett's test. Differences were considered statistically significant when the probability of error was less than 0.05 (p<0.05).

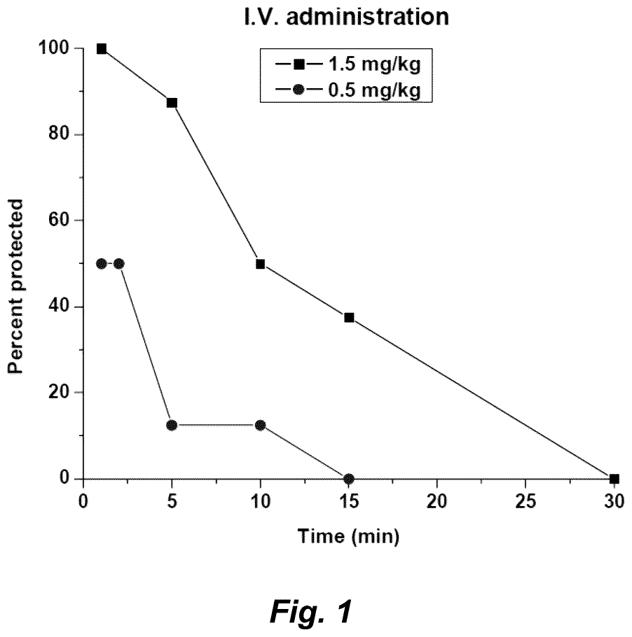

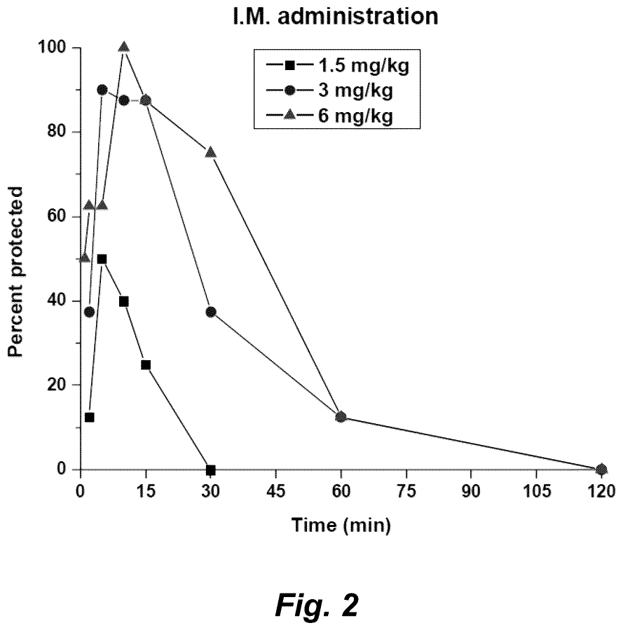

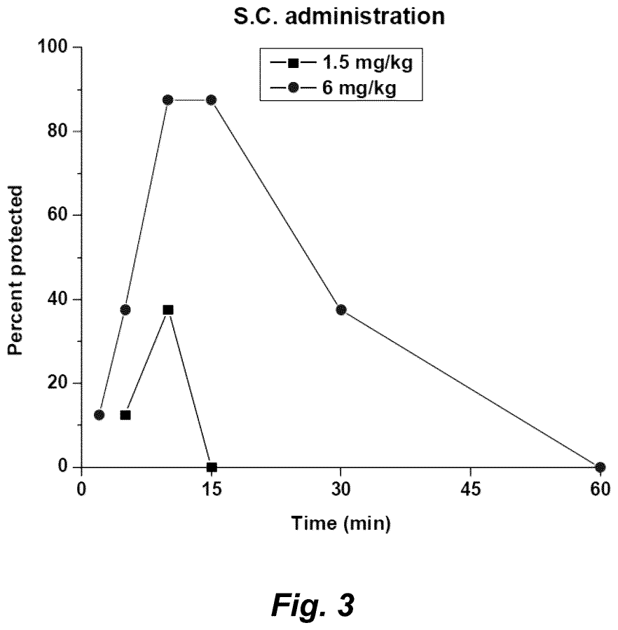

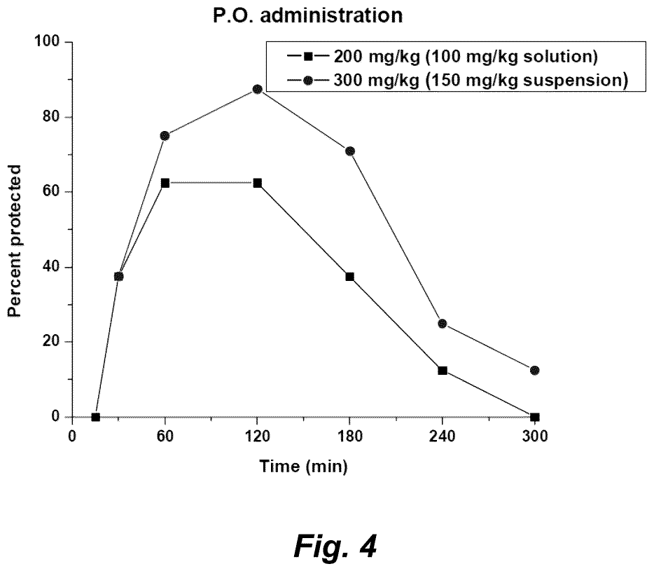

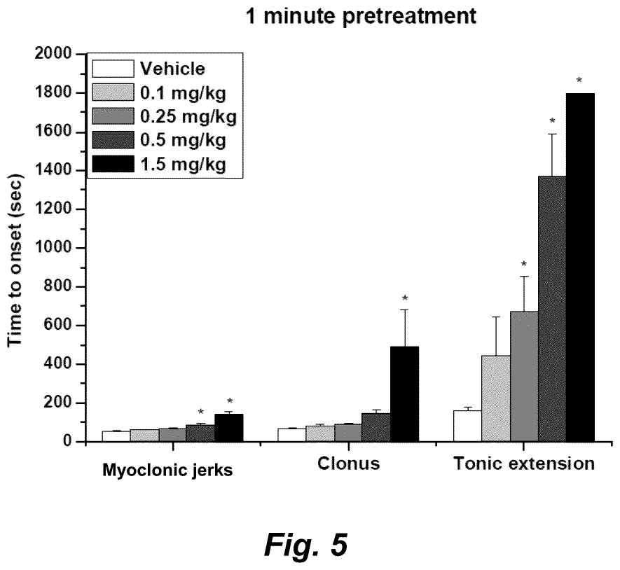

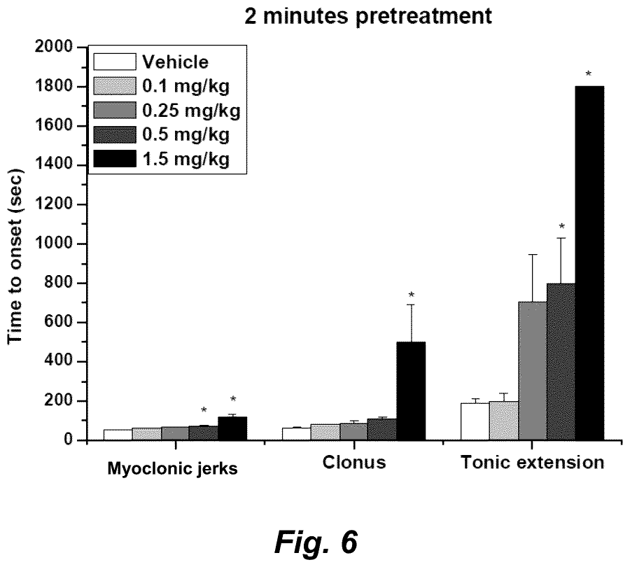

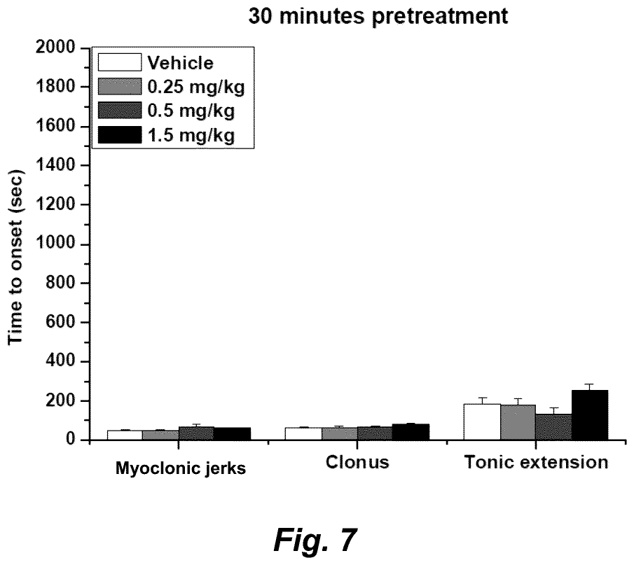

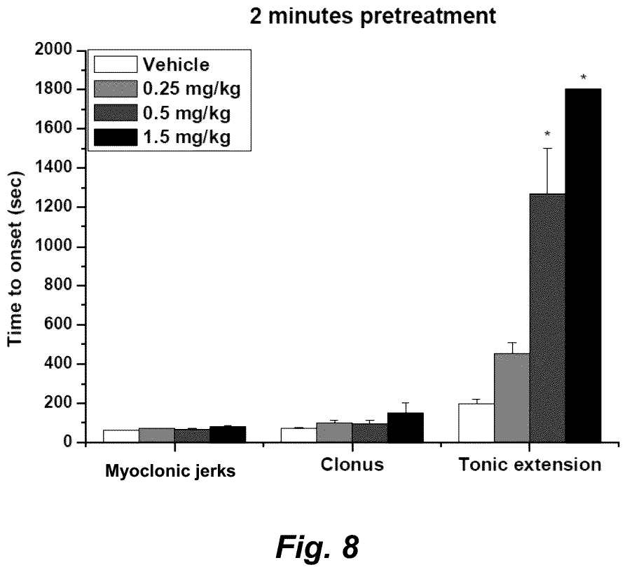

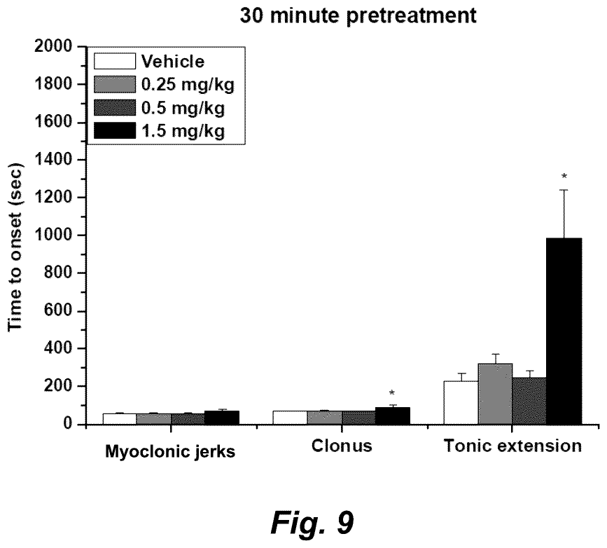

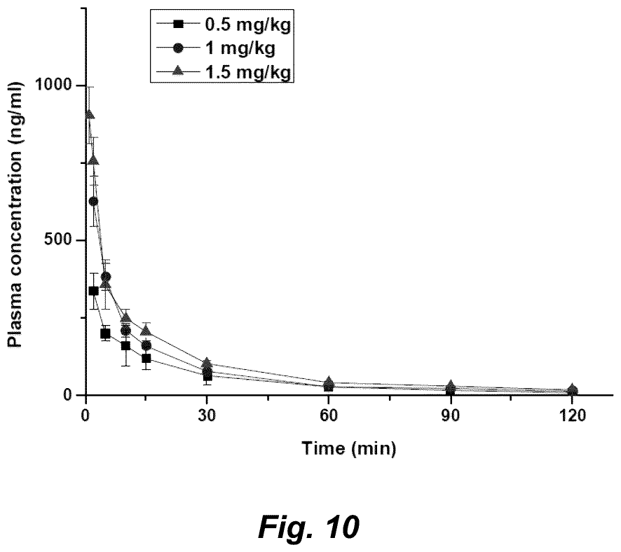

Results are shown in FIGS. 1-10. Our results demonstrate that i.v. 5α,3α-P provides very rapid but transitory anticonvulsant activity. When injected i.m., 5α,3α-P acts comparably quickly and has a longer duration of action. Low bioavailability of 5α,3α-P after oral administration prolongs the time of the peak effect and duration of action. Parenteral 5α,3α-P is useful for the acute treatment of seizures.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of treating, reducing, and/or mitigating symptoms associated with and/or caused by traumatic brain injury, Alzheimer's disease, epilepsy, anxiety, fragile X syndrome, post-traumatic stress disorder, lysosomal storage disorders (Niemann-Pick type C disease), depression (including post-partum depression), premenstrual dysphoric disorder, alcohol craving, and smoking cessation in a subject in need thereof, comprising administration to the subject a steroid.

2. A method of treating, reducing, and/or mitigating symptoms associated with and/or caused by epilepsy, in a subject in need thereof, comprising administration to the subject a steroid.

3. A method of accelerating the termination or abortion of an impending seizure in a subject in need thereof, comprising administration to the subject a steroid.

4. The method of any one of claims 1 to 3, wherein the steroid is a neurosteroid.

5. The method of any one of claims 1 to 4, wherein the neurosteroid is selected from the group consisting of allopregnanolone, allotetrahydrodeoxycorticosterone, ganaxolone, alphaxolone, alphadolone, hydroxydione, minaxolone, and Althesin.

6. The method of claim 5, wherein the neurosteroid is allopregnanolone.

7. The method of any one of claims 5 to 6, wherein the steroid is formulated in a cyclodextrin.

8. The method of any one of claims 1 to 7, wherein the steroid is formulated in hydroxypropyl-beta-cyclodextrin, sulfobutylether-beta-cyclodextrin sodium salt, or a mixture thereof.

9. The method of any one of claims 1 to 8, wherein the subject is experiencing aura.

10. The method of any one of claims 1 to 9, wherein the subject has been warned of an impending seizure.

11. The method of any one of claims 1 to 10, wherein the subject is experiencing a seizure.

12. The method of any one of claims 1 to 11, wherein the subject has status epilepticus.

13. The method of any one of claims 1 to 11, wherein the subject has myoclonic epilepsy.

14. The method of any one of claims 1 to 11, wherein the subject suffers from seizure clusters.

15. The method of any one of claims 1 to 14, wherein the seizure is a tonic seizure.

16. The method of any one of claims 1 to 14, wherein the seizure is a clonic seizure.

17. The method of any one of claims 1 to 16, wherein the subject is a human.

18. The method of any one of claims 1 to 17, wherein the steroid is administered intramuscularly (i.m.), subcutaneously (s.c.) or intravenously (i.v.).

19. The method of any one of claims 1 to 18, wherein the steroid is administered at a dose in the range of about 0.25 mg/kg to about 15 mg/kg.

20. The method of any one of claims 1 to 19, comprising treating, reducing, and/or mitigating symptoms associated with and/or caused by epilepsy by intramuscularly (i.m.), subcutaneously (s.c.) or intravenously (i.v.) administering allopregnanolone formulated in a sulfobutylether-beta-cyclodextrin sodium salt.

21. The method of any one of claims 1 to 20, wherein the epilepsy is status epilepticus.

22. A composition comprising or consisting essentially of a steroid and a cyclodextrin.

23. The composition of claim 22, wherein the steroid is a neurosteroid.

24. The composition of claim 23, wherein the neurosteroid is selected from the group consisting of allopregnanolone, allotetrahydrodeoxycorticosterone, ganaxolone, alphaxolone, alphadolone, hydroxydione, minaxolone, and Althesin.

25. The composition of any one of claims 22 to 24, wherein the steroid is allopregnanolone.

26. The composition of any one of claims 22 to 25, wherein the cyclodextrin is hydroxypropyl-beta-cyclodextrin, sulfobutylether-beta-cyclodextrin sodium salt, or a mixture thereof.

27. The composition of any one of claims 22 to 26, wherein the composition is formulated for intramuscular (i.m.), subcutaneous (s.c.) or intravenous (i.v.) administration.

28. The composition of any one of claims 22 to 27, wherein the composition comprises allopregnanolone and sulfobutylether-beta-cyclodextrin sodium salt.
